(19) United States
(12) Patent Application Publication
(10) Pub. No.: US 2024/0091392 A1
(43) Pub. Date: Mar. 21, 2024

(54) ENDOSCOPIC DEVICE FOR TREATMENT OF INFECTIONS

(71) Applicant: FREESTYLE PARTNERS LLC, Detroit, MI (US)

(72) Inventors: Jennifer K. Rosen, Detroit, MI (US); Benjamin X. Feeney, Nashville, TN (US); Gregory D. DeGrazia, Birmingham, MI (US)

(73) Assignee: FREESTYLE PARTNERS LLC, Detroit, MI (US)

(21) Appl. No.: 18/521,949

(22) Filed: Nov. 28, 2023

Related U.S. Application Data
(63) Continuation of application No. PCT/US22/36508, filed on Jul. 8, 2022.
Application No. PCT/US22/36508 is a continuation-in-part of application No. 17/379,694, filed on Jul. 19, 2021.
Application No. 17/379,694 is a continuation-in-part of application No. 17/333,558, filed on May 28, 2021.
Application No. 17/333,558 is a continuation-in-part of application No. 17/333,565, filed on May 28, 2021.
Application No. 17/333,565 is a continuation of application No. 16/279,253, filed on Feb. 19, 2019, now Pat. No. 11,135,324.
(60) Provisional application No. 62/694,483, filed on Jul. 6, 2018.
(60) Provisional application No. 62/632,716, filed on Feb. 20, 2018.

(51) Int. Cl. A61L 2/00

(52) U.S. Cl. CPC A61L 2/0052; A61L 2202/11; A61L 2202/14; A61L 2202/16

(57) ABSTRACT

An endoscopic irradiation device for inactivating pathogens is disclosed. The device includes a control element and a probe that is directed by the control element. A source of UVC light providing a peak irradiation of about 222 nm is integrated with at least one of the probes and the control element. An attenuating element attenuates irradiation above about 235 nm is located in a path of UVC light generated by the source of UVC light. The attenuating element is integrated with at least one of said medical probe and said control element. In one embodiment, attenuating element takes the form of a band pass filter placed between the source of UVC light and biological tissue being irradiated by the UVC light. Alternatively, the attenuating element is a dopant disposed within a crystalline structure of a light emitting diode that restricts or attenuates transmission above about 235 nm.

### PRIOR APPLICATIONS

The present application claims priority as a continuation application of Patent Cooperation Treaty Application No. PCT/US2022/036508 which was filed on Jul. 8, 2022, which claims priority as a continuation-in-part application to U.S. patent application Ser. No. 17/379,694 filed Jul. 19, 2021, which claims priority to U.S. Pat. No. 11,071,799 filed Mar. 5, 2020, which claims priority to U.S. provisional application Ser. No. 62/963,682, filed Jan. 21, 2020, each of which are hereby incorporated herein by reference in its entirety. The present application is also a continuation-in-part of U.S. patent application Ser. No. 17/333,558, filed May 28, 2021, which claims priority to U.S. Pat. No. 11,020,501 filed on Dec. 11, 2020, which claims priority to U.S. Pat. No. 11,071,799 filed on Mar. 5, 2020, which claims priority to U.S. provisional application Ser. No. 62/963,682 filed Jan. 21, 2020, all of which are hereby incorporated herein by reference in their entireties. The present application is also a continuation-in-part of U.S. patent application Ser. No. 17/333,565, which is a continuation of U.S. Pat. No. 11,135,324, filed Feb. 19, 2019, which claims priority to U.S. provisional application Ser. No. 62/694,482, filed Jul. 6, 2018, and U.S. provisional application Ser. No. 62/632,716, filed Feb. 20, 2018, all of which incorporated herein by reference in their entireties.

### TECHNICAL FIELD

The present invention relates generally toward a device used for in activating bacteria and other pathogens. More specifically, the present invention relates toward an endoscopic device making use of ultraviolet light to inactivate infections found on internal tissue of a human being or other an animal

### BACKGROUND

It is known that infected tissue found on human beings and other animals have been treated with aggressive doses of antibiotics. While antibiotics have proven quite effective in resolving infections, adverse side effects to antibiotics are becoming ever more prevalent. In fact, the use of antibiotics has been so successful that it is widely thought antibiotics have been over-prescribed resulting in antibiotic resistant pathogens.

Excessive use of antibiotics has even led to antibiotic resistant bacteria that is causing alarm within the medical field. Furthermore, antibiotics are also known to kill good and bad bacteria alike. Often the loss of good bacteria leads to adverse physical ailments requiring even further use of antibiotics. Generally, bacterial infections are localized in various tissues, such as, for example the sinus, bladder, colon, liver, to name a few. However, to eradicate localized bacteria, oral or intravenous antibiotics spread throughout a full anatomy sometimes leading to adverse side effects.

It has been thought that treating a bacterial infection in a localized manner would eliminate the adverse side effects of antibiotics. One thought is to use various wavelengths of ultraviolet light to eradicate bacterial infections. The use of UVA, UVB and UVC light has been proposed by inserting a device into an animal and attempting to irradiate infected tissue. However, these broad ranging wavelengths are known to alter DNA of tissue leading to growth of cancerous cells. Furthermore, currently known devices that are contemplated to irradiate infected internal tissue do not provide necessary elements to assure any form of control.

Therefore, it would be desirable to provide a device that enables locally treatment of infected tissue without causing adverse medical issues and providing necessary control to assure proper treatment has been achieved.

### SUMMARY

An endoscopic irradiation device for inactivating pathogens is disclosed. The device includes a control element and a probe that is directed by the control element. A source of UVC light providing a peak irradiation of about 222 nm is integrated with at least one of the probes and the control element. An attenuating element attenuates irradiation above about 235 nm and is located in a path of UVC light generated by the source of UVC light. The attenuating element is integrated with at least one of said medical probe and said control element. In one embodiment, attenuating element takes the form of a band pass filter placed between the source of UVC light and biological tissue being irradiated by the UVC light. Alternatively, the attenuating element is a dopant disposed within a crystalline structure of a light emitting diode that restricts or attenuates transmission above about 235 nm.

The device of the present invention provides the safe and affective doses of light to a localized area of infected tissue without the adverse side effects of antibiotics and unsafe light that is known to cause growth of cancerous tissue. The use of a probe enables transmission of light at the location of infection inside a human being or another animal body. For example, attenuating light having a peak irradiance of about 222 nm while attenuating transmission above 235 nm enable safe eradication or inactivation of bacterial or viral infections inside an animal body. The device may be used simultaneously with endoscopic surgery for disinfecting surgical locations inside the animal body. One nonlimiting example is ablation of a cancerous tumor on the kidney. Subsequent to, or concurrently with, the ablation, the device of the present invention may be inserted into the body cavity to irradiate the ablation site reducing probability of infection. Of particular importance is surgery conducted on a colon or digestive tract where E coli could become problematic. The device of the present invention is well suited to eradicate E coli and any bacteria or other harmful pathogens that are prevalent from colon surgery.

The device of the present invention may also be used for treatment of bacterial or viral infections not related to surgery. For example, the probe may be inserted through a patient mouth and into a patient stomach to inactivate, for example, bacterial and viral infections, such as Klebsiella, Salmonella, Shigella flexneri, Proteus, Enterobacter, Enterococcus faecalis, Enterococcus faecium, Staphylococcus epidermidis, Staphylococcus aureus and Candida albicans, Helicobacter pylori (known to cause ulcers), Clostridium botulinum.

In addition, rectal use of the device of the present invention may be used to local inactivate pathogens in the colon or small intestine such as Clostridium difficile, norovirus, roto virus, Adenovirus, Cytomegalovirus, E. Coli, Shigellosis, Clostridium difficile and Salmonella. Diverticulitis resulting in infection may also be treated. Still further, parasites disposed anywhere in a digestive track may be inactivated through irradiation by the device of the present invention.

Respiratory tract infections may also be treated through irradiation using the probe to provide UV light to an esophagus, bronchial tubes, and lungs of a patient. Sinus infections may also be treated through insertion of the probe. While antibiotics are not known to be affective with respect to viral respiratory infections, the use of UV light could be well suited to reduce viral load in the respiratory system due to the ability of UV light to inactivate viruses.

The uses set forth above are merely exemplary and it is contemplated that many more uses to provide localized eradication of pathogens inside an animal body are within the scope of this emerging technology. Localized administration of the UVC light having a peak irradiance of around 222 nm while attenuating transmission above about 235 nanometers provides a safe and effective method of reducing reliance on oversubscription of antibiotics also providing a more rapid elimination and eradication of infections inside an animal body.

### BRIEF DESCRIPTION OF THE DRAWINGS

Other advantages of the present invention will be readily appreciated, as the same becomes better understood by reference to the following detailed description when considered in connection with the accompanying drawings, wherein:

FIG. 1 shows a first embodiment of the device of the present invention;

FIG. 2 shows an expanded view of the distal end of the probe;

FIG. 3 shows plan view of the light emitting module located at the distal end of the probe

FIG. 4 shows a side sectional view of the light emitting module located at the distal end of the probe;

FIG. 5A-5D shows alternative embodiments of the attenuating element;

FIG. 6A-6B shows alternative embodiments of the optical element used with the light emitting diode and the fiber optic cables;

FIG. 7 shows an alternative embodiment of the probe;

FIG. 8 shows the probe inserted into a patient abdomen to irradiate infected tissue.

FIG. 9 shows an alternative embodiment of the probe having fiber optic cables that irradiate UVC light radially outwardly;

FIG. 10 shows an alternative embodiment of the device of the present invention including a krypton chloride lamp; and

FIG. 11 shows a further alternative embodiment including a probe that is separable from the control element.

### DETAILED DESCRIPTION

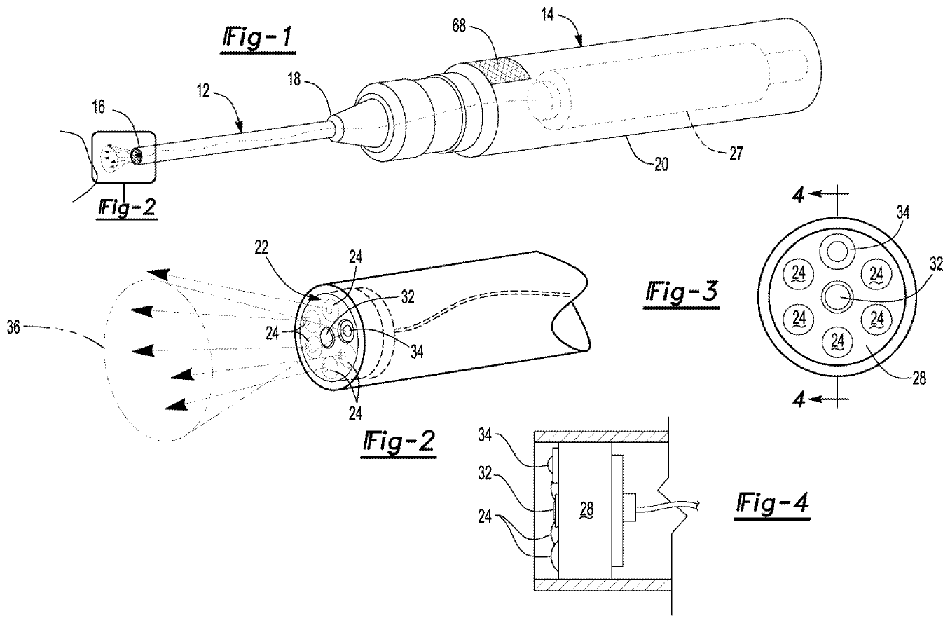

Referring to FIG. 1, an endoscopic device of the present invention is generally shown at 10. The device 10 includes a probe 12 and a control element 14. In one embodiment, the probe 12 includes a distal end 16 and a proximal end 18 that is interconnected with the control element 14. In this embodiment, the control element 14 is defined as a handle 20 for a surgeon, medical specialist to grip during use, or a robotic manipulator. Therefore, the control element 14 enables a user to direct the probe 12 in a manner that will be described further herein below. The device 10 is contemplated to be used either independently from other endoscopic devices or in combination with other endoscopic devices.

The device 10 includes a source 22 of UVC light that provides a peak irradiation of about 222 nm also referred to as Far-UVC light. In one embodiment, the source 22 of UVC light emits Far-UVC light for inactivating pathogens found on internal tissues and organs of a human beings or other animals. The source 22 of UVC light takes the form of either a light emitting diode 24 (LED) or a Krypton chloride lamp 26 (FIG. 10). In another embodiment, the LED 24 is located in the probe 12 And the Krypton chloride lamp 26 is located at the control element 14. Because the LED 24 is more compact than the Krypton chloride lamp 26 it is feasible to locate the LED 24 at the distal end 16 of the probe 12 as best shown in FIGS. 1 and 2. However, it is also feasible to locate the LED 24 at the control element 14. The Krypton chloride lamp 26 is significantly bigger than a typical LED 24 and is more suitable to be placed had the control element 14. The source 22 of UVC light may be powered by a rechargeable energy cell 27 located within the control element 14 or by way of electrical connection to a power outlet.

The LED 24 is included as part of an LED module 28. In this embodiment, a plurality of LED's 24 is integrated with the LED module 28 that is located at the distal end 16 a tubular member 30 that defines the probe 12. The tubular member 30 may take any shape including cylindrical so long as wiring and capable of delivering an electrical charge or signal to the distal end 16 is achieved. As such, the term “tubular” is not limiting as to shape or configuration of the probe 12. The LED module 28 also includes and imaging device 32 for imaging inside the animal body when the device 10 is in use providing the practitioner a view of the tissue or organ being irradiated. The imaging device provides a visible image by way of a same circuit as does the endoscopic cameras used by the medical professionals. In addition, the module 28 include a visible light source 34 for generated visible light to identify and area of irradiation by projecting, for example, a visible indicia 36 on the tissue or organ. This concept is further defined in U.S. Pat. No. 11,071,799, the contents of which are included in their entirety herein by reference.

An attenuating element 48 is included to attenuate UVC light transmission above 235 nm. Alternatively, the attenuating element 48 attenuates UVC light transmission above 230 nm. It is believed even low amounts of transmission of UVC light above 235 nm (or 230 nm) causes transformation of animal tissue resulting in the formation of cancerous cells. Therefore, the combination of transmission of Far-UVC light and an attenuating element renders the use of UVC light safe for irradiating pathogens on body organs and other internal tissue of animals.

When the LED 24 has been implemented, the use of a dopant within the lattice structure of the LED 24 functions as the attenuating element 48. In one embodiment, the dopant functions as a bandpass filter absorbing UVC light above 235 nm (or 230 nm) when the LED lattice emits Far-UVC light. Alternatively, the dopant modifies irradiation of the LED lattice preventing irradiation above 235 nm (or 230 nm).

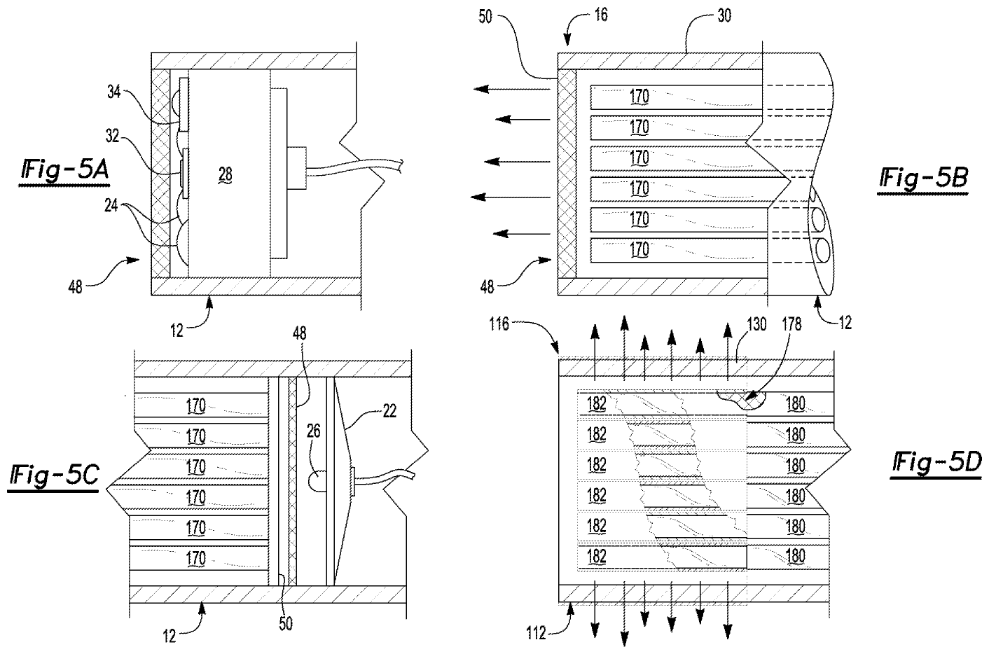

Alternatively, the attenuating element 48 is a band pass filter 50 disposed in the path of irradiation generated by the LED 24 as represented in FIG. 5. Therefore, it may not be necessary for the LED lattice to include a dopant specifically for attenuating irradiation above 235 nm (or 230 nm) because the attenuating element 48 in the form of the band pass filter 50 serves this function. However, it should be understood by those of ordinary skill in the art that the attenuating element 48 may include both a dopant hand a bandpass filter 50.

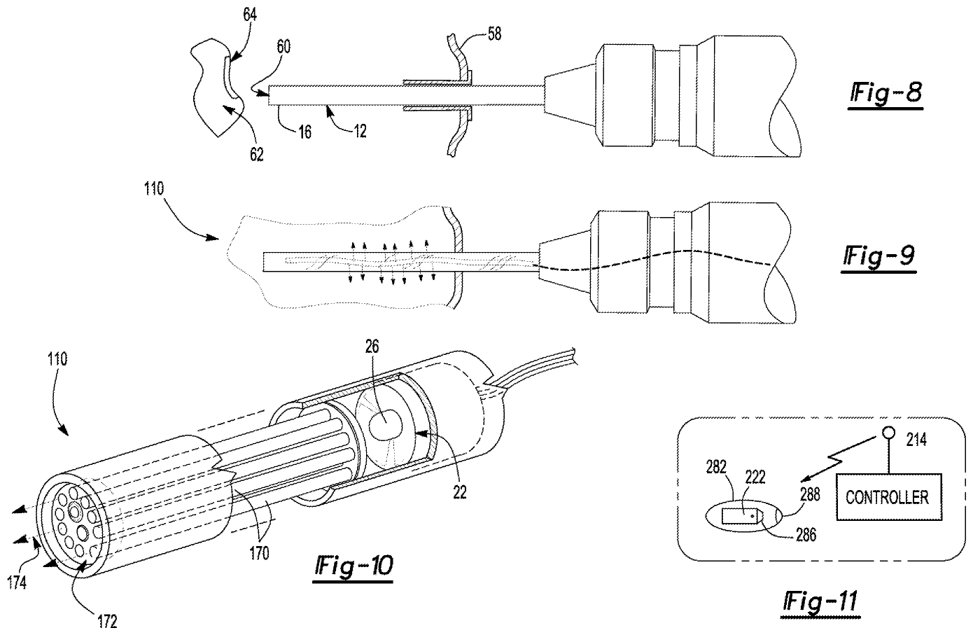

As represented in FIG. 6, an optical element 52 may be included to modify the UVC light being transmitted by the source 22 of UVC light. In this embodiment, the optical element takes the form of a lens configure to focus or diverge light transmitted by the source 22. The optical element 52 may take the form of any one of, (i) a plano-convex lens, (ii) a bi-convex lens, (iii) a plano-concave lens, (iv) a bi-concave lens, (v) a positive meniscus lens, and (vi) a negative meniscus lens and a collimator. In certain situations, the optical element 52 may generate a focal point or focal line on the tissue being irradiated to reduce surface area of irradiation or increase energy transfer to the tissue. Alternatively, the optical element 52 generates divergent light increasing a surface area of illumination on the tissue by the source 22 of light. This concept is further defined by U.S. Pat. No. 11,071,799 the contents of which are included herein by reference in their entirety. It should be understood that the imaging device 32 and the visible light source 34 may be independent from the module 28 for providing a view toward the tissue 62 (FIG. 8) without being subject to distortions related to the attenuating element 48 or the optical element 52.

In one embodiment, the optical element 52 is formulated to also act as a bandpass filter. Alternatively, the optical element 52 works in combination with the attenuating element 48 whether the attenuating element is a bandpass filter 50 or a dopant. In this instance, the optical element 52 and the bandpass filter 50 or disposed in a layered configuration disposed in the path of UVC light.

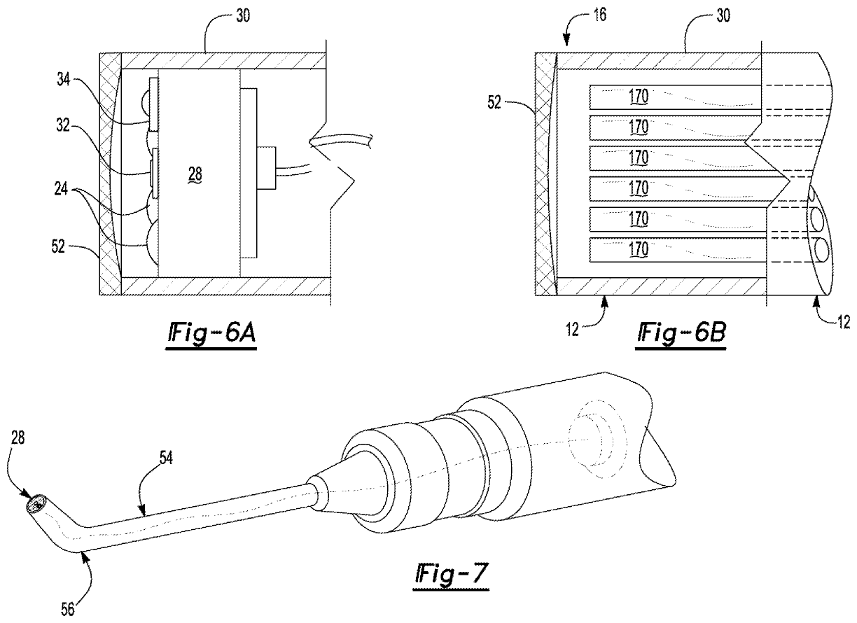

FIG. 7 shows an alternative probe 54 used in circumstances where it is desirable to provide irradiation at a direction offset from an axis defined by the probe. In this embodiment, the alternative probe defines an elbow 56 that redirects the distal end 16 of the alternative probe 54 to an angle proximal end 18 and tubular wall 56 immediately extending therefrom. This configuration is particularly useful for irradiating infected tissue, on, by way of nonlimiting example, colon walls, intestine walls, stomach lining, respiratory systems, and sinuses by providing directed irradiation.

FIG. 8 shows the device 10 being inserted into an abdomen through a patient abdomen wall 58. An endoscopic sleeve 60 is first inserted into an opening in the abdomen to assist movement of the probe 12 in the abdomen 58 to a desired location. The endoscopic sleeve 60 receives the probe 12 providing a smooth surface for the probe to easily move into and out of the patient abdomen 58. As such, the distal end 16 of the probe 12 is easily moved toward an organ 62 or other tissue having an infected area 64.

In this way, the LED 24 may be placed at a predetermined distance proximate the infected area 64 enabling optimal irradiation of the infected area 64. It should be understood that the probe 12 includes a distance sensor 66 that may be integrated with the LED module 28 or separate from the module 28 to detect distance of the LED 24 from the infected area 62. The sensor 62 it is interconnected by way of a controller or equivalent to an indicator 68 to signal the practitioner optimal distance has been achieved and irradiation of the infected area of 62 may now begin.

A further embodiment of the device is shown in FIGS. 9 and 10 generally at 110. The embodiment of the alternative device 110 is described with respect to irradiation being generated by the Krypton chloride lamp 26. However, the LED 24 may also be suitable for generating irradiation for the alternative device 110. In this embodiment, the Krypton chloride lamp 26 is located in the control element 14.

The Krypton chloride lamp provides UVC illumination to a bundle of fiber optic cables 170. The fiber optic cables 170 transmit the UVC light in a known manner to an optical module 172 located at a distal end 116 for the probe 112. The optical module 172 orients the fiber optic cables 170 to direct UVC irradiation in an axially relative to the probe 112 and along arrows 174. It is believed that the irradiation from the ends of the fiber optic cables 170 achieve more directed irradiation than does the LED 124 due to the non-coherent nature of the UVC light. Furthermore, the use of Krypton chloride excimer technology is believed to irradiate at higher energy UVC light than presently available LED technology providing a possibility of more rapid inactivation of pathogens on internal tissue.

It is known that Krypton chloride produces Far-UVC light having a peak irradiance at about 222 nm. However, irradiation is also generated above 230 nm at levels that are unsafe for human or other animal exposure. Therefore, as set forth above, it is desirable to include an attenuating element 148 as is explained hereinabove. Referring to FIG. 5B, the attenuating element 148 is located at the distal end 116 of the probe 112 in the path of the irradiation emitted at the ends of the fiber optic cables 170. Thus, Far-UVC light generated by the Krypton chloride lamp 26 is not attenuated by the bandpass filter 150 until exiting the ends of the fiber optic cables 170.

Alternatively, the attenuating element 148 is located adjacent to, or proximate to krypton chloride lamp 26 in the path of the UVC irradiation as is shown in FIG. 5C. Thus, Far-UVC light above 230 nm (or 235 nm) is attenuated prior to traveling through the fiber optic cables 170. In one embodiment, the fiber optic cables 170 include a tubular outer surface 176 and the Far-UVC light is prevented from passing through the outer surface 176 so that no loss of irradiance is experience when the Far-UVC light exits the end of the cable 170. Thus, to diffuse or focus the Far-UVC light, an optical element 152 may be placed at the distal end 116 of the probe 112 independent of the attenuating element 148 as is shown in FIG. 6B. Alternatively, each of the fiber optic cables 170 are oriented at the distal end 116 of the probe 112 to direct UVC light toward a single focal point to maximize irradiation energy at that point.

In a further embodiment, the fiber optic cables 170 include a translucent section 178 so that the Far-UVC light radiates radially outwardly as shown in FIG. 9. Thus, large sections of, for example, colon, small intestine, stomach, respiratory and sinuses may be irradiated for inactivating bacterial or viral infections that are wide reaching. The fiber optic cables 170 include an opaque section 180 that prevents light from radiating radially outwardly while transmitting light in an axial direction relative to the cables 170 in a known manner. The opaque section 180 is located proximate to the Krypton chloride lamp 26 while the translucent section 178 extends through the probe 112 toward the distal end 116.

In this embodiment, the tubular member 130 of the probe 112 is necessarily translucent to allow the UVC light to reach the infected tissue of the patient. As shown in FIG. 5C, and explained above, the attenuating element 148 may be located adjacent the source 122 of the UVC light to attenuate light above 230 nm (or 235 nm) is attenuated prior to entering the fiber optic cables 170. Alternatively, as shown in FIG. 5D, an alternative attenuating element 182 covers one of the translucent section 178 of the fiber optic cable 170 or the tubular member 130 of the probe 112.

A further alternative embodiment is shown in FIG. 11 generally at 210 where like elements to the first embodiment include like element numbers but in the 200 series. In this embodiment a probe 212 is separable from a control element 214. The probe 212 takes the form of an irradiation module 282 defined by a translucent capsule 284. A source of UVC light 222 is disposed inside the translucent capsule 284 along with a power source 286. The power source is activated and deactivated by a control element 214. The source 222 of UVC light includes peak irradiance of around 222 nm and an attenuating element to attenuate irradiation above 230 nm (or 235 nm). It is contemplated that the source 222 of UVC light in this embodiment is an LED 224 with a dopant to attenuate excessive irradiation. However, the translucent capsule 284 may also include the attenuating element either by way of a coating or chemical integration.

The alternative probe 212 is designed to be swallowed by a patient or inserted into patient abdomen or organ for localized irradiation of infected tissue. It is further contemplated that the alternative probe 212 is retained inside a patient for an extended period to administer cyclical doses of UVC light to infected tissue. A single alternative probe 212 or a plurality of probes 212 may be used as necessary to treat infected tissue.

The alternative probe 212 may be inserted and manipulated into position by an endoscopic instrument. Alternatively, the alternative probe 212 may include an independent motion module 288 that may be operated by the control element 214 or through machine learning software. The power source 286 is chargeable by way of magnetic resonance and magnetic induction in similar manner as is a pacemaker and other devices retained within a patient for extended periods of time.

The invention has been described in an illustrative manner; many modifications and variations of the present invention are possible. It is therefore to be understood that within the specification, the reference numerals are merely for convenience, and are not to be in any way limiting, and that the invention may be practiced otherwise than is specifically described. Therefore, the invention can be practiced otherwise than is specifically described within the scope of the stated claims following this first disclosed embodiment.

What is claimed is:

1. An endoscopic irradiation device for inactivating pathogens, comprising:
a control element;
an endoscopic probe being directed by said control element:
a source of UVC light providing a peak irradiation of about 222 nm, said source of UVC light being integrated with at least one of said endoscopic probe and said control element; and
an attenuating element attenuating irradiation above about 235 nm located in a path of UVC light generated by said source of UVC light and being integrated with at least one of said endoscopic probe and said control element.

2. The device set forth in claim 1, wherein said attenuating element comprises a band pass filter.

3. The device set forth in claim 1, wherein said attenuating element comprises a light emitting diode being configured to restrict transmission of UVC light above about 235 nm.

4. The device set forth in claim 3, wherein said light emitting diode is located at said endoscopic probe.

5. The device set forth in claim 4, wherein said light emitting diode transmits UVC light in a radially outward direction from said endoscopic probe.

6. The device set forth in claim 1, wherein said endoscopic probe includes optical fibers for transmitting UVC light generated at a location spaced from said endoscopic probe.

7. The device set forth in claim 6, wherein said optical fibers transmit UVC light in a radially outward direction from said endoscopic probe.

8. The device set forth in claim 6, wherein said optical fibers transmit UVC light substantially unidirectionally.

9. The device set forth in claim 1, wherein said attenuating element comprises a band pass filter located at said source of UVC light.

10. The device set forth in claim 6, wherein said wherein said attenuating element comprises a band pass filter located at said optical fibers.

11. The device set forth in claim 1, wherein said source of UVC light comprises a light emitting diode and said attenuating element comprises a dopant included in a chemical lattice defining said light emitting diode for attenuating transmission of UVC light above 235 nm.

12. The device set forth in claim 1, wherein said endoscopic probe comprises a drone being separable from said controller and said controller directs said drone from a remote location.

13. The device set forth in claim 12, wherein said drone includes light emitting diode and said attenuating element comprises a dopant for attenuating transmission of UVC light above 235 nm.

14. The device set forth in claim 1, wherein said endoscopic probe defines a distal end including a visible light source for transmitting visible light for identifying a location on tissue disposed inside a human being and an animal body being irradiated by the source of UVC light.

15. The device set forth in claim 14, wherein said distal end of said endoscopic probe includes an imaging device for providing a medical professional an image of the tissue being illuminated by the visible light generated-by the visible light source.

16. The device set forth in claim 1, wherein said endoscopic probe includes a distance sensor for identifying distance between the source of UVC light and tissue being irradiated by said UVC light.

17. The device set forth in claim 1, wherein said endoscopic probe defines a distal end including an optical element comprising one of (i) a plano-convex lens, (ii) a bi-convex lens, (iii) a plano-concave lens, (iv) a bi-concave lens, (v) a positive meniscus lens, and (vi) a negative meniscus lens and a collimator for modifying irradiation area of the UVC light.
